(19) United States
(12) Patent Application Publication
(10) Pub. No.: US 2004/0122380 A1
(43) Pub. Date: Jun. 24, 2004

(54) Blunt cannula with bent tip

(76) Inventor: David S. Utterberg, Seattle, WA (US)

Correspondence Address: SEYFARTH SHAW, 55 EAST MONROE STREET, SUITE 4200, CHICAGO, IL, 60603-5803, US

(21) Appl. No.: 10/326,335

(22) Filed: Dec. 19, 2002

(51) Int. Cl. A61M 5/32

(52) U.S. Cl. 604/272

(57) ABSTRACT

A cannula is provided for medical use, and a method for manufacture. The cannula comprises a rigid tube having inner and outer surfaces and having a beveled end. The beveled end defines an end surface which is joined to the inner and outer surfaces of the tube at inner and outer junction edges. The end surface is typically flat in transverse direction between the inner and outer junction edges, and the outer end defines an outer end portion that is bent transversely inwardly.

### BACKGROUND OF THE INVENTION

In the area of hemodialysis and other forms of blood processing, the various older techniques for gaining access to the bloodstream of a patient have exhibited significant, known, technical disadvantages. In response to this, a new technology for blood access, for example, has been developed by Vasca, Inc. of Topsfield, Mass., involving an implantible port chamber in the body of a patient, which is used in a manner described in PCT publications WO98/31416 and WO99/03527, as well as in other references. Other, similar technology exists as well. Specifically, an implanted port is implanted within the skin of the patient, with a preformed needle track or “tract” extending from the entrance of the port through the tissue and through the skin. Thus, repeated penetration of the needle or tissue “tract” by an access cannula can take place without cutting tissue, since cells similar to scar tissue form around at least some of the walls of the tissue tract, providing a substantially permanent tract similar to the tissue tract passing through earlobes in the case of pierced ears.

The use of such a “buttonhole” permanent tract for vascular access has been pioneered by Zbylut J. Twardowski, and is discussed in the literature.

In accordance with this invention, a cannula is provided having a beveled, open tip which is particularly suitable for penetration of such a needle tract while minimizing the cutting of tissue and the creation of pain as the needle is inserted through the skin and within the tract, typically into communication with an implanted port.

### DESCRIPTION OF THE INVENTION

In accordance with this invention, a cannula for medical use is provided, which cannula comprises: a rigid tube, typically of the proportions of a surgical metal fistula needle for hemodialysis.

The needle or cannula used in this invention has inner and outer surfaces, and also had a beveled end. The beveled end defines an angled end surface. The end surface joins to the inner and outer surfaces of the tube at inner and outer, typically dull, junction edges, with the end surface being typically generally flat in transverse direction between the inner and outer junction edges, where, generally, conventional hypodermic needles have sharp junction edges. Furthermore, the beveled end defines an outer end portion that is bent transversely inwardly, preferably in a smooth, arcuate manner.

Thus, the needle of this invention typically is free of sharp edges that are sufficiently sharp to cut tissue, contrary for example to the needle of Tersteegen et al., U.S. Pat. No. 4,368,738, which shows a bent tip needle expressly for the purposes of cutting and penetrating tissue.

Preferably, the inner and outer junction edges are “broken” in the cannula of this invention. The word “broken” is intended to have the known meaning that is used in machining for an edge or the like that by some process is abraded to lose its sharpness. An angled edge which comprises the junction of two faces of a machine part may be “broken” by being subject to abrasion of some kind, so that the sharp, angled edge is rendered dull. A machined steel block having a precisely 90° angled lines is actually capable of cutting fingers unless the edge is “broken,” to reduce or eliminate the capacity for cutting skin.

Typically, it is preferred that the junction edges of the cannula are dull enough so that the junction edges do not readily cut skin in normal use.

In some embodiments, an inner portion of the beveled cannula end is unbent, with the unbent portion defining an angle of about 20°-45° to the cannula longitudinal axis.

The cannula of this invention may typically have a sidewall which has a wall thickness of about 0.05 to 0.16 mm, which is a normal dimension for hypodermic needles for blood access.

Preferably, the outer end portion of the beveled end is smoothly bent transversely inwardly, without a sharply angled transition point of bending. Also, the outer end may define an arcuate outer surface. Such a cannula, having relatively blunt edges, and having an inwardly turned outer end, can penetrate through a pre-cut “buttonhole” needle tract in a patient, with minimal further cutting of tissue. The inwardly turned end in particular tends to guide and center the cannula along the needle tract so that the tip does not dig into the wall. Also as the cannula advances, the outer surface of the inwardly turned end receives a centering lateral vector force that counteracts lateral vector forces that act on the remaining surfaces of the cannula end, as discussed below, to center the advancing cannula. The cannula advances, spreading the wall of the needle tract apart, but with minimal cutting of tissue, to relatively painlessly enter into communication with an implanted port, which, in turn, typically communicates with the vascular system of the patient.

Furthermore, some cannula embodiments may carry a conventional back-eye aperture adjacent to the end.

A method of communication with an internal access site under the skin of the patient is thus provided. This method comprises: providing a preformed access channel through the skin to the internal access site; and advancing through the access channel a rigid cannula having a beveled front end with dull edges. The beveled front end defines an outer end portion that is bent transversely inwardly toward the longitudinal axis of the cannula. Such a cannula can be better centered in the access channel to reduce tissue abrasion resulting from such advancement.

Typically, the cannula is flow connected to flexible tubing, which can be part of an extracorporeal bloodflow set such as arterial or venous sets for hemodialysis. It is also generally preferred for the outer end portion of the needle to be smoothly bent transversely inwardly without a sharply angled transition point.

Thus, a cannula for medical use is described which can penetrate preformed needle tracts extending through the skin of a patient, for access to an implanted port, for example, which may be in connection with the vascular system of a patient, or which may be for other purposes.

Specifically, an internal access site under the skin of the patient may be brought into the communication with a tubular set for extracorporeal blood processing or other clinical use by providing a preformed access channel through the skin to the internal access site. This preformed internal access site, commonly called a “buttonhole”, may be provided when access to an interior access site within the patient is needed on a regular, chronic basis, for example as in hemodialysis.

In accordance with this invention, one advances through the access channel a rigid cannula having a beveled front end with dull edges. The beveled front end defines an outer end portion that is bent transversely inwardly toward the longitudinal axis of the cannula preferably terminating before the outer point of the beveled end crosses the longitudinal axis.

Because of this, the advancing cannula is better centered in the access channel, having the effect of reducing tissue abrasion which results from the advancement. Thus, a preformed access channel can heal to an extent, with scar tissue cells surrounding much of its length so that abrasion and cutting of tissue is avoided, while access to the vascular system of a patient, for example, can be regularly achieved on an intermittent basis. The cannula is typically flow connected to flexible tubing as in an arterial or a venous set for an extracorporeal blood handling process such as hemodialysis, pheresis, or another form of chronically repeated blood treatment or collection.

### DESCRIPTION OF DRAWINGS

In the drawings, FIG. 1 is an enlarged, perspective view of a cannula manufactured in accordance with this invention.

FIG. 2 is a fragmentary, plan view of the beveled end of the cannula of FIG. 1.

FIG. 3 is a sectional view taken along line 3-3 of FIG. 2, but rotated counterclockwise by 90°.

FIG. 4 is an enlarged, longitudinal sectional view of the beveled end of the cannula of FIG. 1, shown as being advanced through a “buttonhole” needle tract to an implanted port for vascular access.

FIG. 5 is an enlarged, fragmentary, perspective view of the beveled end of the cannula of FIG. 1.

FIG. 6 is generally exploded, schematic view of a package containing the cannula of this invention.

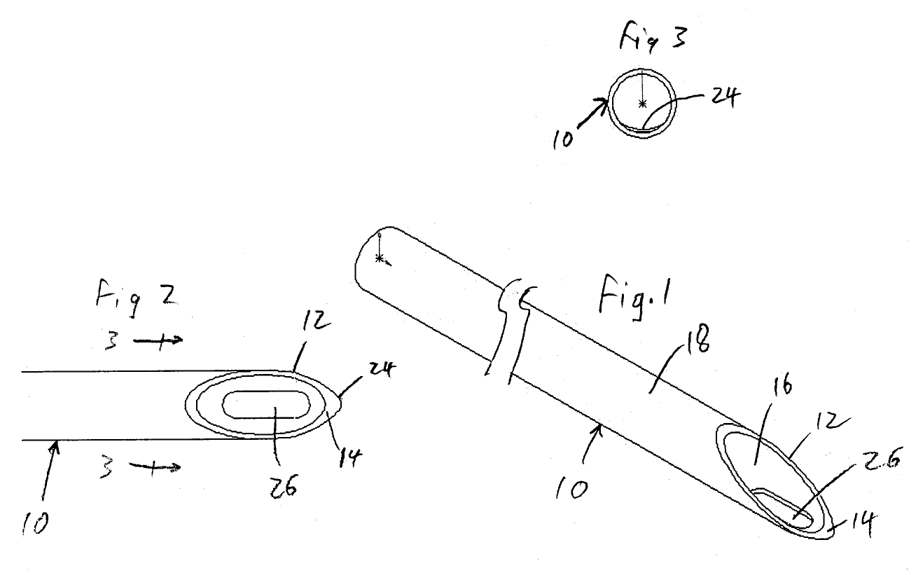

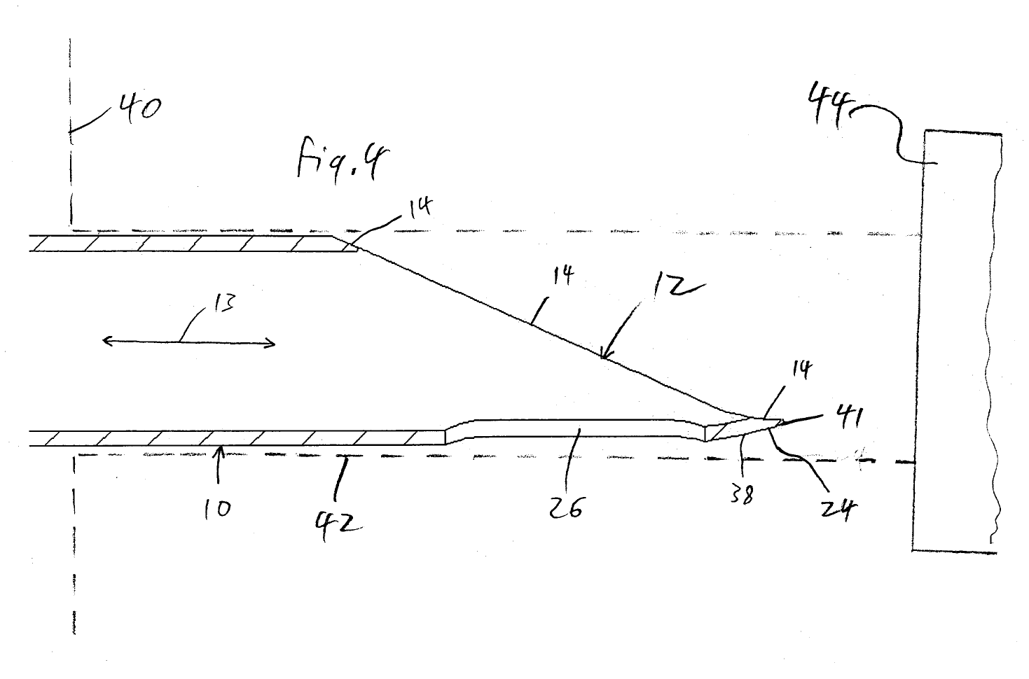

### DESCRIPTION OF SPECIFIC EMBODIMENTS

Referring to the drawings, cannula 10 is shown, typically comprising a surgical steel tube 18 of typical size for hypodermic needles, particularly, needles used in dialysis. These cannulae typically have a sidewall with a wall thickness of essentially 0.05-0.16 mm., with the other dimensions corresponding to that dimension. Cannula 10 may be mounted in a hub 52 (FIG. 6) in conventional manner that may connect to a tubular set 50 or a syringe in any conventional way. Also, the hub may be of an angled type as shown in U.S. Pat. No. 6,267,750 entitled Set with Angled Needle.

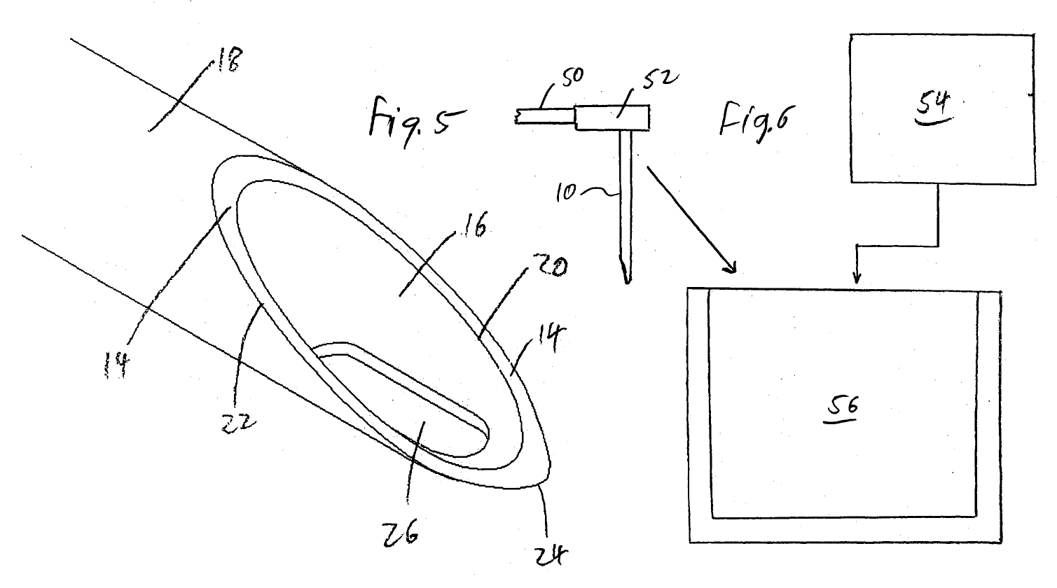

A squared, perpendicular end of a cannula tube precursor may be straight cut on an angle, in a conventional first step for manufacturing hypodermic cannulae, to form beveled end 12, having an angle in this specific embodiment of about 30° to the longitudinal axis 13 of the tube, as shown in FIG. 4. This cut forms a beveled end 12, which defines end surface 14 which, in turn defines a closed, generally oval loop, as shown in FIG. 5. End surface 14 joins to the inner tube surface 16 and the outer tube surface 18 of the cannula tube to define inner junction edge 20 and outer junction edge 22, each of which edges also-comprise a generally oval-loop. Between junction edges 20, 22, end surface 14 is generally flat in the transverse direction between the inner and outer junction edges 20, 22, as particularly illustrated in FIGS. 4 and 5. In other words, edges 20 and 22 are preferably positioned along the cannula 10 at the same longitudinal position without significant longitudinal spacing to make one edge 20, 22 lead the other. Particularly, outer edge 22 should not lead inner edge 20, since that would contribute to tissue abrasion as the needle advances through a “buttonhole” tract. Inner edge 20 may lead outer edge 22, if desired.

By a process step typically subsequent to the cutting of beveled surface 14, an outer end portion 24 of the cannula beveled end is shown to be bent transversely inwardly from the normal planar configuration of surface 14 that is formed by the initial (and typically only) straight bevel cut used in the manufacture of this embodiment.

Cannula 10 in this embodiment also defines a backeye 26, as may be conventionally and optionally found in various needle tip designs.

Thus a blunt cannula is provided, particularly for easy insertion into a needle tract extending through the skin for repeated insertion, while minimizing the further cutting of tissue and the consequent pain experienced by the patient. The blunt cannula also has the upturned end 24 to further reduce the cutting or abrasion of tissue as the cannula is advanced, and to assist in centering the cannula as it advances. The centering is facilitated because the outer surface 38 of the needle tip is acted on by forces that tend to urge the needle tip (as shown in FIG. 4) upwardly as the needle advances. This tends to counteract lateral forces exerted in the opposite direction upon surface 14 as the cannula is advanced. This is particularly desirable, in that it tends to prevent needle point 41 from abrading particularly the fat layer portion of the skin as the needle penetrates, on a repeated basis, day by day, for access to the vascular system for hemodialysis or another medical procedure. Also, this is advantageous in that the entire needle tract that extends through the skin can be protected, which is particularly needed before scar tissue forms in the whole needle tract.

Thus it can be seen that the cannula of this invention is protective of the tissue surrounding a “buttonhole” tract that provides cannula access to the interior of a patient's body, for purposes such as access to an implanted access port for the vascular system.

Referring again to FIG. 4, needle 10 of this invention is shown to be advanced through the skin 40 of a patient, passing through a “buttonhole”-type needle tract 42 which has been preformed, so that the respective dull edges 41, 14 of the needle is involved in very little cutting of tissue as the needle advances, to reduce pain and injury to the patient. In this embodiment, needle 10 is advanced into engagement with a conventional, implanted port 44, which communicates in valved manner with the vascular system of a patient, for example as described in the previously cited PCT publications. As needle 10 advances, the forces acting on angled surface 38 can counteract forces that are acting on surface 14, to provide better needle centering as the needle advances.

FIG. 6 shows cannula 10, carried in perpendicular manner to connected flexible set tubing 50 by means of hub 52. the needle and set tubing, plus instructions for use 54 may be carried in conventional manner by sterile packaging 56, with needle and set tubing 10, 50 being sealed inside the packaging 56.

The above has been offered for illustrative purposes only, and is not intended to limit the invention of this application, which is as defined in the claims below.

That which is claimed is:

1. A cannula for medical use, which comprises:
a rigid tube having inner and outer surface and having a beveled end, said beveled end defining an end surface, said end surface joining to the inner and outer surfaces of said tube at inner and outer junction edges, said beveled end being dull enough so that the junction edges do not readily cut skin in normal use, said beveled end having an outer end portion that is bent transversely inwardly.

2. The cannula of claim 1 in which said inner and outer junction edges are broken.

3. The cannula of claim 1 in which an inner portion of said beveled end is unbent, said unbent portion defining an angle of 20-45° to the cannula longitudinal axis.

4. The cannula of claim 3 in which said angle is essentially 30°.

5. The cannula of claim 1 which has a side wall with a wall thickness of essentially 0.05 to 0.16 mm.

6. The cannula of claim 1 in which said end surface is generally flat in transverse direction between said inner and outer junction edges.

7. The cannula of claim 1 which is dull enough at said beveled end to not penetrate adult skin at a pressure of 100 gm.

8. The cannula of claim 7 which has a side wall with a wall thickness of essentially 0.05 to 0.16 mm.

9. The cannula of claim 7 in which a backeye is formed adjacent to said end surface.

10. The cannula of claim 7 in which said outer end portion is smoothly bent transversely inwardly without a sharply angled transition point.

11. The cannula of claim 7 in which said outer end defines an arcuate outer surface.

12. The cannula of claim 7 in which a portion of said beveled end is unbent, said unbent portion defining an angle of 20-45° to the cannula longitudinal axis.

13. A kit comprising:
the cannula of claim 1;
instructions for use of the cannula with a patient; and
a package containing the cannula and the instructions for use.

14. The cannula of claim 1 in which said inwardly bent outer end portion ends prior to crossing the cannula longitudinal axis.

15. The method of communication with an internal access site under the skin of a patient, which comprises:
providing a preformed access channel through the skin to the internal access site;
advancing through said access channel a rigid cannula having a beveled front end with dull edges, said beveled front end defining an outer end portion that is bent transversely inwardly toward the longitudinal axis of said cannula, whereby said advancing cannula is better centered in said access channel to reduce tissue abrasion resulting from said advancement.

16. The method of claim 15 in which said cannula is flow connected to flexible tubing.

17. The method of claim 16 in which said outer end portion is smoothly bent transversely inwardly without a sharply angled transition point.

18. The method of claim 15 in which said outer end portion is smoothly bent transversely inwardly without a sharply angled transition point.
